(19) United States
(12) Patent Application Publication
(10) Pub. No.: US 2008/0008727 A1
(43) Pub. Date: Jan. 10, 2008

(54) Compositions comprising adapalene dissolved with cyclodextrins

(75) Inventors: Laurent Fredon, Roquefort Les Pins (FR); Sandrine Orsoni, Mandelieu (FR); Agnes Ferrandis, Mougins (FR); Claire Mallard, Mougins (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 11/812,889

(22) Filed: Jun. 22, 2007

Related U.S. Application Data
(63) Continuation of application No. PCT/FR05/03173, filed on Dec. 16, 2005.

(30) Foreign Application Priority Data
Dec. 22, 2004 (FR) 0413760

(51) Int. Cl. A61K 31/702; A61K 9/00; A61K 9/107; A61P 17/00; A61Q 19/00

(52) U.S. Cl. 424/401; 424/400; 514/25

(57) ABSTRACT

Cosmetic/pharmaceutical compositions comprising adapalene dissolved in an aqueous medium with cyclodextrin and/or derivatives thereof, notably as inclusion complexes therewith, are useful as cosmetics and for the therapeutic treatment, e.g., of acne.

### CROSS-REFERENCE TO PRIORITY/PCT APPLICATIONS

This application claims priority under 35 U.S.C. § 119 of FR 0413760, filed Dec. 22, 2004, and is a continuation of PCT/FR 2005/003173, filed Dec. 16, 2005 and designating the United States (published in the French language on Jul. 6, 2006 as WO 2006/070093 A1; the title and abstract were also published in English), each hereby expressly incorporated by reference in its entirety and each assigned to the assignee hereof.

### BACKGROUND OF THE INVENTION

1. Technical Field of the Invention

The present invention relates to compositions for cosmetic or pharmaceutical applications comprising adapalene (6-[3-(1-adamantyl)-4-methoxyphenyl]-2-naphthoic acid) in an aqueous medium and cyclodextrins or cyclodextrin derivatives, said adapalene preferably being dissolved in the form of complexes with the cyclodextrins or derivatives thereof.

2. Description of Background and/or Related and/or Prior Art

As adapalene is a compound that is practically insoluble in water, it is generally dispersed in compositions of gel or cream type (0.1% DIFFERINE® gel and 0.1% DIFFERINE® cream). The present invention describes, on the contrary, a technique for dissolving the adapalene in aqueous medium that enables it to be dissolved in aqueous topical compositions and in particular at a content of 0.1% by weight relative to the total weight of the composition (w/w) or 0.3% w/w.

In the prior art (U.S. Pat. No. 4,371,673) it is proposed to increase the solubility of retinoic acid in aqueous media by forming complexes using cyclodextrins or derivatives thereof.

A recent study on this subject carried out by Anadolu et al., with patients suffering from acne vulgaris (“Improved efficacy and tolerability of retinoic acid in acne vulgaris: a new topical formulation with cyclodextrin complex”, European Academy of Dermatology and Venereology JEADV (2004) 18,416-421), reports a more effective acne treatment with retinoic acid complexed with β-cyclodextrins relative to a reference product marketed under the trademark RETINO FORTE® gel 0.05% w/w.

EP-0,486,395 discloses an aqueous gel based on retinoic acid and hydroxypropyl-β-cyclodextrin.

WO 2004/084883 describes the use of a complex of isotretinoin with cyclodextrins for preparing a pharmaceutical composition for oral administration.

Adapalene is recognized as being a medication that is as effective as tretinoin for treating acne, but that has the advantage of causing fewer adverse affects than tretinoin; which makes this a product of choice.

There is therefore a need to prepare a topical pharmaceutical composition containing adapalene, the formulation of which is stable, well tolerated and which optimizes the penetration of adapalene into the skin.

### SUMMARY OF THE INVENTION

It has now surprisingly been demonstrated that a formulation as described herein enables adapalene to be dissolved and gives good results for tolerance and chemical stability at room temperature, with an improved penetration into the skin. Thus, this enables the amount of active principle administered to be decreased, where appropriate.

The formulations presented in the examples below show that dissolving adapalene using cyclodextrins is stable over time from a chemical point of view.

Furthermore, dissolving adapalene in an aqueous medium in the form of complexes improves the cutaneous penetration of the active principle while still having very good cutaneous tolerance.

A novel process has also been developed for formulating the compositions according to the invention and therefore for dissolving adapalene in an aqueous medium.

The present invention therefore features compositions comprising, in a physiologically acceptable aqueous medium:
a) adapalene; and
b) one or more cyclodextrins or cyclodextrin derivatives.

### BRIEF DESCRIPTION OF THE DRAWINGS

FIG. 1 is a graph showing the result of the area under the curves (AVC) from the 2nd and the 22nd days for skin oedemas on the inner face of the ear of mice for the various products tested, and

FIG. 2 is a graph showing the kinetics of the average thickness of the mouse ears from the 2nd and 22nd days for the various products tested.

### DETAILED DESCRIPTION OF BEST MODE AND SPECIFIC/PREFERRED EMBODIMENTS OF THE INVENTION

The compositions according to the invention are in the form of a homogeneous solution. The term “homogeneous solution” means a solution that does not contain adapalene in the form of crystals.

The compositions according to the invention comprise, as active principle, either adapalene alone (6-[3-(1-adamantyl)-4-methoxyphenyl]-2-napthoic acid), or one of its precursors and/or derivatives, or adapalene or one of its precursors and/or derivatives in association or in combination with another active agent.

The term “adapalene derivatives” is understood especially to mean its esters and its salts, such as the salts formed with a pharmaceutically acceptable base, especially mineral bases such as sodium hydroxide, potassium hydroxide and ammonium hydroxide or organic bases such as lysine, arginine, or N-methylglucamine.

The term “adapalene salts” is also understood to mean the salts formed with fatty amines such as dioctylamine and stearylamine.

Moreover, the active agent that can be used in association or in combination with adapalene may be an antibiotic, such as doxycycline or clindamycin. Adapalene may also be associated or combined with benzoyl peroxide.

In the present invention, adapalene or one of its precursors and/or derivatives is present in solution in the aqueous medium.

Preferably, the compositions according to the invention comprise, in a physiologically acceptable aqueous medium:
a) adapalene; and
b) one or more cyclodextrins or cyclodextrin derivatives,
   in which said compound a) is dissolved in the form of complexes with one or more cyclodextrins or cyclodextrin derivatives.

The term “physiologically acceptable aqueous medium” means a medium that is compatible with the skin, the mucous membranes and/or the appendages that is well known to a person skilled in the art.

The cyclodextrins used in the present invention are those known from the literature.

The cyclodextrins (CDs) are cyclic oligosaccharides composed of (α-1,4)α-D-glucopyranose units with a central lipophilic cavity and an external hydrophilic surface (Frömming K H, Szejtli J: “Cyclodextrins in pharmacy”, Kluwer Academic Publishers, Dortrecht, 1994).

Cyclodextrins are known to increase the solubility of molecules by forming a “cage”-shaped structure having an external hydrophilic part and an internal hydrophobic part. Cyclodextrins may thus form inclusion complexes with many medications by accepting the whole molecule, or more commonly the lipophilic part of the molecule, inside the cavity.

The most abundant natural cyclodextrins are α-cyclodextrins, β-cyclodextrins and γ-cyclodextrins.

The α-cyclodextrins (also known under the trademark Schardinger's α-dextrin, cyclomaltohexaose, cyclohexaglucan, cyclohexaamylose, α-CD, ACD, C6A) comprise 6 glucopyranose units. The β-cyclodextrins (also known under the trademark Schardinger's β-dextrin, cyclomaltoheptaose, cycloheptaglucan, cycloheptaamylose, β-CD, BCD, C7A) comprise 7 glucopyranose units and the γ-cyclodextrins (also known under the trademark Schardinger's γ-dextrin, cyclomaltooctaose, cyclooctaglucan, cyclooctaamylose, γ-CD, GCD, C8A) comprise 8 glucopyranose units.

Among these three types of CDs, β-cyclodextrins appear to be the most useful as complexing pharmaceutical agents due to the size of their cavity, their availability, their properties and their low cost.

According to Dr J. Szejtli (“Cyclodextrins”, in Encyclopedia of Supramolecular Chemistry, Eds. Marcel Dekker, 2004) the cyclodextrins are advantageous but also have limiting factors that restrict the application of cyclodextrins to certain types of pharmaceutical products. Furthermore, not all products are suitable for complexing with cyclodextrins. Many products cannot be complexed or else complexing does not provide any basic advantage. Inorganic compounds are generally unsuitable for complexing with cyclodextrins.

Cyclodextrin derivatives are also used in the present invention. In cyclodextrins, each glucopyranose unit has three free hydroxyl groups that differ in their function and their reactivity.

The term “cyclodextrin derivative” means a cyclodextrin of which all or some of the hydroxyl groups have been modified by substitution of the hydroxyl group or of the hydrogen atom.

Ester, ether, anhydro, deoxy-, acidic, basic, etc. derivatives may be prepared by chemical or enzymatic reactions well known to one skilled in the art.

For example, in β-CDs, 21 hydroxyl groups may be modified by substituting the hydrogen atom or the hydroxyl group with a wide variety of groups such as alkyl, hydroxyalkyl, carboxyalkyl, amino, thio, tosyl, glucosyl, maltosyl, etc. groups.

Among preferred derivatives, exemplary are the derivatives of α-cyclodextrins, p-cyclodextrins, γ-cyclodextrins and in particular the methyl derivatives of cyclodextrins and 2-hydroxypropyl-β-cyclodextrin (HPCD); 2-hydroxyethyl-β-cyclodextrin; 2-hydroxypropyl-γ-cyclodextrin and 2-hydroxyethyl-γ-cyclodextrin. In particular, the HPCD marketed especially under the trademark KLEPTOSE HPB® by Roquette is exemplary.

From the expression “adapalene in the form of complexes with cyclodextrins” it should be understood that the active adapalene molecule, whatever its conformation, is totally or partially included inside the “cage” structure.

In general, the inclusion complexes with cyclodextrins according to the present invention are prepared by conventional methods, well known to one skilled in the art. The formation of the complex may be facilitated by suitable adjustment of the pH or the use of solvents, such as organic solvents like methanol or ethanol

In the complex obtained according to the present invention, the ratio from adapalene and the cyclodextrins is from 1/1 to 1/1000, this ratio preferably being from 1/1 to 1/20.

In the present text, unless otherwise stated, it is understood that when the concentration ranges are given, they include the upper and lower limits of said range.

According to one embodiment of the invention, the compositions comprise from 1% to 60% (w/w) of cyclodextrins or cyclodextrin derivatives and comprises from 0.01% to 1% (w/w) of adapalene.

The compositions according to the invention preferentially comprise from 3% to 40% (w/w) of cyclodextrins or cyclodextrin derivatives and comprise from 0.05% to 0.5% (w/w) of adapalene. Even more preferably, they comprise from 3% to 15% (w/w), preferentially from 5% to 15% of cyclodextrins or cyclodextrin derivatives, and from 0.1% to 0.3% (w/w) of adapalene. Preferentially, the compositions comprise 0.1% (w/w) of adapalene. Alternatively, the compositions preferentially comprise 0.3% (w/w) of adapalene.

Optionally, the compositions comprise a basifying agent. The term “basifying agent” means an agent able to increase the pH of the composition. In particular, the compositions may comprise from 0% to 1% of one or more basifying agents. Preferably, the compositions comprise from 0.02% to 0.5% of one or more basifying agents.

Among such basifying agents, exemplary are inorganic bases with inorganic hydroxides, inorganic oxides and the salts of weak inorganic acids. By way of example, representative is sodium hydroxide. As other such basifying agents, exemplary are organic bases such as 2-amino-2-methyl-1-propanol (AMP) and primary, monosubstituted, or disubstituted amines, nitriles and isocyanides, amides, aromatic and non-aromatic amino heterocycles with, by way of non-limiting example, triethanolamine, cyclohexylamine, or piperidine. Preferred are AMP and sodium hydroxide as basifying agent.

The compositions according to the invention may also comprise, formulated into a pharmacologically acceptable medium, one or more of the following ingredients:
a) one or more gelling agents;
b) one or more surfactants;
c) one or more fatty phases;
d) one or more preservatives; and
e) one or more emollients/humectants.

The term “pharmacologically acceptable medium” means a medium that is compatible with the skin, the mucous membranes and/or the appendages.

Among these gelling agents, exemplary are the carbomers marketed under the generic trademark CARBOPOL®, the carbomers said to be insensitive to electrolytes, marketed under the trademark ULTREZ 10® or CARBOPOL ETD® by BF Goodrich, polysaccharides with, by way of non-limiting example, xanthan gum such as KELTROL T® marketed by Kelco, guar gum, chitosans, cellulose and its derivatives such as hydroxyethyl cellulose, such as the product marketed under the trademark NATROSOL HHX 250® by Aqualon, and the copolymer of sodium acrylamide and acrylamino-2-methylpropanesulfonate as a 40% dispersion in isohexadecane and the polysorbate 80 marketed under the trademark SIMULGEL 600® by Seppic.

As the preferred gelling agent, exemplary is the hydroxyethyl cellulose marketed especially under the trademark NATROSOL HHX 250®.

Among the surfactants, exemplary are the ionic surfactants such as sodium lauryl sulfate. Also exemplary are the non-ionic surfactants such as, for example, the polysorbate 80 marketed under the trademark TWEEN 80®.

The fatty phases of the composition according to the invention may comprise, for example, plant, mineral, animal or synthetic oils, silicone oils, and mixtures thereof.

As examples of mineral oils, representative are the paraffin oils of various viscosities such as PRIMOL 352®, MARCOL 82®, MARCOL 152® marketed by Esso.

As plant oils, exemplary are sweet almond oil, palm oil, soybean oil, sesame oil and sunflower oil.

As animal oils, exemplary are lanolin, squalene, fish oil and mink oil.

As synthetic oils, exemplary are an ester such as cetearyl isononanoate, such as the product marketed under the trademark CETIOL SN® by Cognis France, diisopropyl adipate such as the product marketed under the trademark CERAPHYL 230® by ISF, isopropyl palmitate such as the product marketed under the trademark CRODAMOL IPP® by Croda, caprylic/capric triglyceride such as MIGLYOL 812® marketed by Univar.

As silicone oils, exemplary are a dimethicone such as the product marketed under the trademark Dow Corning 200 Fluid®, a cyclomethicone such as the product marketed under the trademark Dow Corning 244 Fluid® by Dow Corning or the product marketed under the trademark MIRASIL CM5® by SACI-CFPA.

Solid fatty substances such as natural or synthetic waxes could also be used. In this case, one skilled in the art will adjust the preparation heating temperature depending on the presence or absence of these solids.

Examples of preservatives include benzoic acid and its derivatives with benzyl alcohol, benzalkonium chloride, sodium benzoate, bronopol, chlorohexidine, chlorocresol and its derivatives, ethyl alcohol, phenethyl alcohol, phenoxyethanol, potassium sorbate, diazolidinyl urea, parabens, or mixtures thereof. Methylparaben and phenoxyethanol are particularly preferred.

Examples of humectants/emollients include glycerol, sorbitol, and propylene glycol.

The compositions may comprise, in addition, additives usually used in the cosmetics or pharmaceutical field, such as a humectant and/or co-solvent, a soothing agent, an antioxidant, a chelating agent, one or more wetting surfactants, one or more neutralizing agents and mixtures thereof.

Of course, one skilled in the art will take care to select this or these optional additional compounds, and/or their quantity, such that the advantageous properties of the compositions according to the invention are not, or not substantially, changed.

These additives may be present in the composition in an amount from 0.001% to 20% by weight relative to the total weight of the composition.

Anti-irritants and/or “soothing” agents may also be added to the formulations, such as strontium nitrate, shea butter, 18β-glycyrrhetinic acid (enoxolone) and its potassium or zinc salts, α-tocopherol acetate, allantoin, aloe vera, α-bisabolol, talc and mixtures thereof.

In a particular embodiment of the invention, the composition comprises:
from 0.05% to 1% of adapalene;
from 3% to 40% of one or more cyclodextrins or derivatives;
from 0.01% to 2% of one or more gelling agents;
from 0% to 1% of one or more basifying agents; and
from 0.01% to 2% of one or more preservatives;
   and in a preferred embodiment, the composition comprises:
from 0.1% to 0.3% of adapalene;
from 5% to 15% of one or more cyclodextrins or derivatives;
from 0.1% to 1.5% of one or more gelling agents;
from 0.02% to 0.5% of one or more basifying agents; and
from 0.01% to 1.5% of one or more preservatives.

Advantageously, the composition according to the invention is in aqueous form and in particular in the form of a gel, a cream-gel, a cream, an emulsion, a lotion, a spray or a mousse.

The present invention also features the compositions as described previously as medications.

This invention also features a process for preparing a composition according to the invention, comprising the following steps:
i) complexing step enabling the active agent to be dissolved: this step comprises the mixing of a solution of one or more cyclodextrins or cyclodextrin derivatives with the active agent, which is at least one compound selected from adapalene, one of its precursors or one of its derivatives, in order to obtain a homogeneous solution;
ii) step for preparing the formulation base: obtaining gel, cream-gel, cream, emulsion, lotion, spray or mousse formulae; and
iii) step for introducing the active agent solution obtained in i) into the formulation base prepared in ii), and obtaining a homogeneous solution.

The two first steps (complexing and preparing the formulation base) take place successively or in parallel: they are independent of one another. On the other hand, the third step (introduction of the active agent solution into the formulation base) is carried out after completion of the first two steps.

According to a particular embodiment of the invention, the process for preparing a composition comprises the following steps:

Phase 1: Complexing Step:
1) preparing a solution of cyclodextrins with purified water;
2) stirring the solution until the cyclodextrins have completely dissolved, around 30 minutes, and a homogeneous solution is obtained;
3) introducing the active agent, such as adapalene or one of its precursors or one of its derivatives, into the preceding solution; and
4) stirring the mixture until the active principle has completely dissolved and a homogeneous solution is obtained.

Alternatively, when a basifying agent is introduced into the solution, this may be introduced right at the beginning of the steps. In this case, it is recommended to prepare a solution of basifying agent with purified water then to magnetically stir the solution so as to obtain a vortex at room temperature. The solution of cyclodextrins is then introduced into this basifying agent/purified water solution. The process as described previously is then followed from point 2).

Phase 2: Preparing the Formulation Base:

Depending on the formulation base selected (gel, cream-gel, cream, lotion, emulsion, spray, mousse) this base is prepared according to the conventional process that is well known to one skilled in the art.

Phase 3: Mixing Phases 1 and 2:
1) pouring the solution obtained in phase 1 into the formulation base obtained in phase 2, and mixing with stirring;
2) decreasing the stirring and leaving the composition obtained in 1) to become homogeneous;
3) adding water if necessary to compensate for the losses due to evaporation; and
4) homogenizing and packaging.

The incorporation of optional additives could take place, depending on their chemical nature, in the course of one of the steps of the preparation process described above.

In particular, the incorporation of preservatives may be carried out at the end of phase 2 or even in the course of phase 3. For example, in the process for obtaining the gel formula, the preservative is introduced during phase 2 after homogenization or even during phase 3 after mixing the phases 1 and 2.

The present invention also features the use of the novel compositions as described previously in cosmetics and in dermatology.

In particular, this invention features the use of a composition as described previously for manufacturing a pharmaceutical preparation intended for treating and/or preventing dermatological conditions linked to a keratinization disorder involving cell differentiation and proliferation, especially for treating acne vulgaris, comedonal or polymorphic acne, acne rosacea, nodulocystic acne, acne conglobata, senial acne, secondary acne such as solar, drug or occupational acne.

The compositions according to the invention are preferentially administered via topical application.

This invention also features a non-therapeutic cosmetic regime or regimen for beautifying the skin and/or improving its surface appearance, wherein a composition comprising adapalene dissolved in an aqueous medium with one or more cyclodextrins or cyclodextrin derivatives, and optionally a sunscreen is applied to the skin and/or appendages.

The sunscreen that can be used is understood to be at least one organic photoprotective agent and/or at least one inorganic photoprotective agent that is active in the UV-A and/or UV-B range (absorbers) and that is water-soluble or liposoluble or else insoluble in the commonly used cosmetic solvents. Exemplary are terephthalylidene dicamphor sulfonic acid, manufactured under the trademark MEXORYL SX by Chimex, and drometrizole trisiloxane, marketed under the trademark SILATRIZOLE by Rhodia Chimie.

The cosmetic compositions according to the invention are administered to treat cutaneous imperfections, dilating pores, inhomogeneous skin texture and/or red blotches.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

In the compositions below (Examples 1, 2 and 4), the amounts of the various constituents are expressed in weight percentages relative to the total weight of the composition. The examples are carried out at room temperature, except where stated otherwise.

### Example 1

### Gel-Type Formulation with AMP as Basifying Agent

| Starting materials | Content (% w/w) |
|---|---|
| AMP | 0.04 |
| Adapalene | 0.10 |
| Hydroxypropyl-β-cyclodextrin | 11.95 |
| Hydroxyethyl cellulose | 1.00 |
| Methylparaben | 0.10 |
| Phenoxyethanol | 0.50 |
| Purified water | qs for 100 |

The formulation was prepared according to the following process:

Phase 1: Complexing Step:
1) A 1% solution of 2-amino-2-methyl-1-propanol (AMP) was prepared with purified water.
2) The purified water, then the aqueous solution of AMP prepared previously were weighed out into a beaker.
3) The mixture was stirred with a magnetic stirrer so as to obtain a vortex at room temperature.
4) The hydroxypropyl-β-cyclodextrin was weighed out and slowly introduced into the preceding solution.
5) The mixture was stirred until the cyclodextrins had completely dissolved (about 30 minutes).
6) The adapalene was weighed out and introduced into the preceding solution.
7) The mixture was stirred until the active principle had completely dissolved and a clear solution was obtained.

Phase 2: Preparation of the Gel:
1) The purified water was weighed out into a beaker, stirred using a Rayneri mixer at 200 rpm and heated up to 85° C. using a hotplate.
2) The methylparaben was weighed out and introduced.
3) The mixture was stirred until the methylparaben had completely dissolved.
4) The beaker was removed from the hotplate and left to cool down to 50° C.
5) The hydroxyethyl cellulose was weighed out and introduced.
6) The mixture was left to homogenize.
7) The stirring speed was adjusted to the consistency of the gel obtained (600 rpm).

Phase 3: Mixing of Phases 1 and 2:
1) Phase 1 was poured into phase 2.
2) The stirring was decreased and the mixture was left to homogenize until a homogeneous gel was obtained.
3) The phenoxyethanol was weighed out and introduced.
4) The mixture was left to homogenize.
5) Water was added if necessary in order to compensate for the losses due to evaporation.
6) The mixture was homogenized and packaged.

The gel obtained was a transparent gel.

### Example 2

### Gel-Type Formulation with Sodium Hydroxide (NaOH) as the Basifying Agent

| Starting materials | Content (% w/w) |
|---|---|
| Sodium hydroxide | 0.02 |
| Adapalene | 0.10 |
| Hydroxypropyl-β-cyclodextrin | 5.80 |
| Hydroxyethyl cellulose | 1.00 |
| Methylparaben | 0.10 |
| Phenoxyethanol | 0.50 |
| Purified water | qs for 100 |

The preparation process was identical to that described in Example 1, with AMP being replaced by sodium hydroxide.

### Example 3

### Chemical Stability of the Gel Formulation According to Example 1

The chemical stability of the gel formulation according to Example 1 was measured by HPLC over three months at room temperature (RT):

| | | |
|---|---|---|
| Chemical | T zero | Room temperature |
| STABILITY | | Adapalene: 101.3% |
| | T 3 months | Room temperature |
| | | Adapalene: 101.2% |

The results show that this composition was chemically stable over three months at room temperature.

### Example 4

### Emulsion-Type Formulation with AMP as the Basifying Agent

| Starting materials | Content (% w/w) |
|---|---|
| AMP | 0.04 |
| Adapalene | 0.10 |
| Hydroxypropyl-β-cyclodextrin | 11.95 |
| PEG-20 methyl glucose sesquistearate | 3.50 |
| Methyl glucose sesquistearate | 3.50 |
| Propylparaben | 0.10 |
| Phenoxyethanol | 0.50 |
| Perhydrosqualene | 6.00 |
| Cyclomethicone-5 | 13.00 |
| Disodium EDTA | 0.10 |
| Hydroxyethyl cellulose | 1.00 |
| Glycerol | 3.00 |
| Methylparaben | 0.10 |
| Purified water | qs for 100 |

The formulation was prepared according to the following process:

Phase 1: Complexing Step:
1) A 1% solution of 2-amino-2-methyl-1-propanol (AMP) was prepared with purified water.
2) The purified water, then the aqueous solution of AMP prepared previously were weighed out into a beaker.
3) The mixture was stirred with a magnetic stirrer so as to obtain a vortex at room temperature.
4) The hydroxypropyl-β-cyclodextrin was weighed out and slowly introduced into the preceding solution.
5) The mixture was stirred until the cyclodextrins had completely dissolved (about 30 minutes).
6) The adapalene was weighed out and introduced into the preceding solution.
7) The mixture was stirred until the active principle had completely dissolved and a clear solution was obtained.

Phase 2: Production of the Emulsion:

Preparation of the fatty phase:
1) A beaker was tared and the excipients of the fatty phase were weighed out: PEG-20 methyl glucose sesquistearate, methyl glucose sesquistearate, propylparaben, phenoxyethanol and perhydrosqualene.
2) The mixture was melted over a water bath at 80° C.
3) When everything had melted, the temperature was allowed to drop to 60° C. and the cyclomethicone-5 was introduced.

Preparation of the Aqueous Phase:
1) A beaker was tared, and the water was weighed out and stirred.
2) The disodium EDTA was weighed out and introduced by sprinkling.
3) The hydroxyethyl cellulose was weighed out and also introduced by sprinkling.
4) The mixture was stirred without heating until the gelling agent had completely dispersed.
5) When the mixture was homogeneous, the aqueous phase was brought to 60° C. over a water bath.

Preparation of the Preservative Phase:
1 ) A small beaker was tared, and the glycerol and methylparaben were weighed out.
2) The beaker was put on a hotplate with magnetic stirring to make the methylparaben melt in the glycerol. The mixture was then completely transparent.
3) This phase was then introduced into the aqueous phase that was previously heated to 60° C.

Emulsification:
1) The fatty phase was stirred on a hotplate at 60° C.
2) The aqueous phase was then very slowly introduced into the fatty phase. The emulsification was carried out without any problems.
3) Heating was maintained for 5 minutes, then the hotplate was removed to let the product slowly cool with gentle stirring.

Phase 3: Mixing of Phases 1 and 2:
1) Phase 1 was poured into phase 2 (the emulsion).
2) The stirring was decreased and the mixture was left to homogenize until a homogeneous emulsion was obtained.
3) The phenoxyethanol was weighed out and introduced.
4) The mixture was left to homogenize.
5) Water was added if necessary in order to compensate for the losses due to evaporation.
6) The mixture was homogenized and packaged.

### Example 5

### Tolerance Study in BALB/c Mice:

The present study was aimed at evaluating the irritant power of the composition according to the invention comprising 0.1% of adapalene, on the skin of the ear of the BALB/c mice after repeated topical application over 6 days.

The daily topical application (20 μl) of the two formulations described in Examples 1 and 2 was carried out on the skin of the ear of BALB/c mice divided into four groups (female mice about 9 weeks old) at a rate of one application per day for 6 days.

The evaluation was carried out by measurements of the thickness of the ear using an Oditest and by clinical observation of the animals from the 2nd to the 12th day then from the 15th to the 19th day and then on the 22nd day.

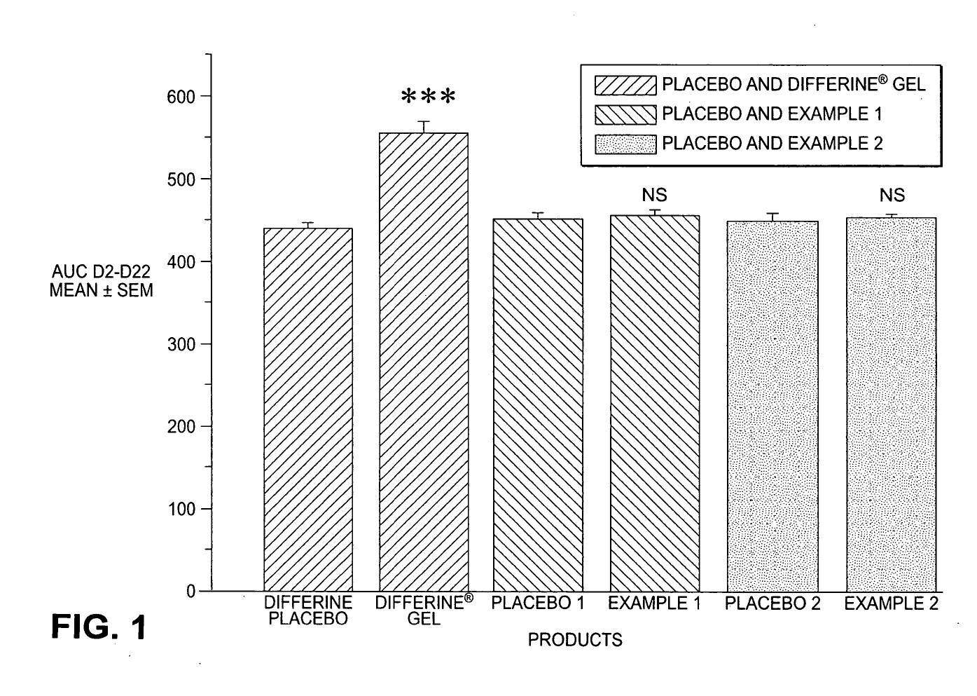

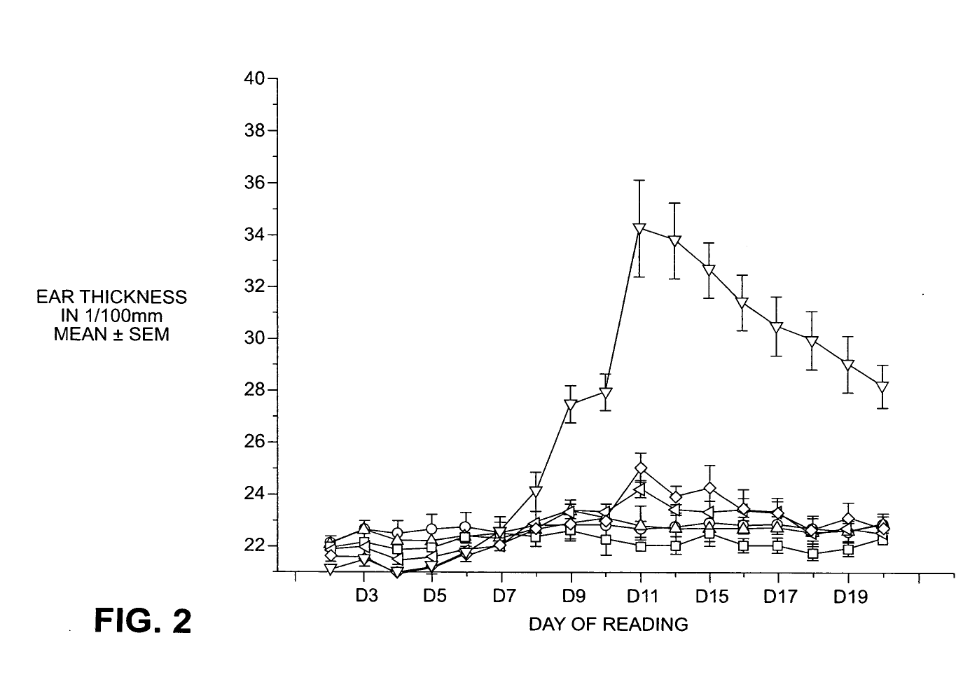

The results are represented in the table below and in FIGS. 1 and 2 where:

FIG. 1 is the result of the areas under the curves (AUC) from the 2nd and the 22nd days for skin oedemas on the inner face of the ear of mice for the various products to be tested, with:
DIFFERINE® gel and its placebo
Adapalene/cyclodextrin (Example 1) and its placebo
Adapalene/cyclodextrin (Example 2) and its placebo

FIG. 2 is the kinetics of the average thickness of the mouse ears from the 2nd and 22nd days for the various products to be tested, with:
DIFFERINE® gel placebo
Cyclodextrin/AMP placebo
Cyclodextrin/NaOH placebo
DIFFERINE® gel
Cyclodextrin formula according to Example 1
Cyclodextrin formula according to Example 2

Summary table of the results of the AUC and maximum increase percentages for the mouse ear thickness:

| | | | Increase | |
|---|---|---|---|---|
| | AUC D2-D22 | | in AUC | Statistics |
| | Mean | Standard | versus | versus |
| | (1/100 mm) | deviation | carrier (%) | carrier |
| 0.1% | 442.5 | 5.3 | | |
| DIFFERINE ® gel | | | | |
| placebo | | | | |
| Cyclodextrin/AMP | 453.3 | 7.6 | | |
| placebo | | | | |
| Cyclodextrin/NaOH | 451.1 | 9.8 | | |
| placebo | | | | |
| DIFFERINE ® gel | 556.8 | 14.1 | 25.8 | *** |
| 0.1% cyclodextrin | 457.6 | 6.8 | −0.9 | NS |
| formula according | | | | |
| to Example 1 | | | | |
| 0.1% cyclodextrin | 454.8 | 4.3 | 0.8 | NS |
| formula according | | | | |
| to Example 2 | | | | |

The results of the study show that after daily 20 μl topical applications of the product to be tested from D1 to D6, on the ear of BALB/c mice:
the DIFFERINE® gel placebo does not cause any irritation;
the cyclodextrin/AMP and cyclodextrin/NaOH placebos do not cause any irritation;
DIFFERINE® gel causes an irritation with an increase of the AUC of 26%, which is significant relative to its placebo; and
the two 0.1% adapalene formulations according to Examples 1 and 2 do not cause any irritation that is significant relative to their respective carriers.

This example demonstrates that the formulae according to the invention have a better in vivo tolerance relative to the reference product 0.1% DIFFERINE® gel.

### Example 6

### In vitro Release Study

The present study aimed to compare the in vitro release and penetration through human skin, in a non-occlusive manner, of adapalene formulated at 0.1% (w/w) in a gel containing cyclodextrins, with the commercial reference product (0.1% DIFFERINE® gel).

In the novel formula, the adapalene was complexed in hydroxypropyl-β-cyclodextrin and formulated into a gel based on hydroxyethyl cellulose (NATROSOL HHX 250®) as described in Example 1.

The experimental conditions were the following: the absorption studies were carried out using excised human skin mounted under static conditions for a period of 16 hours. Three skin samples from women (aged 68 years old) were used.

An amount of 10 mg of each formula (10 μg of adapalene) was applied onto a surface area of 1 cm² of skin. The concentrations of adapalene in the fluid fractions recovered over time and remaining in the skin at the end of the study were evaluated by the HPLC method with fluorescence detection (based on a validated method; limit of quantification: 1 ng/ml).

The experimental results show that, regardless of formulation tested, the adapalene is distributed mainly in the skin (epidermis, including stratum corneum, and dermis). The total amounts that penetrated (stratum corneum +epidermis +dermis +recipient liquid) were:

| | | 0.1% cyclodextrin |
|---|---|---|
| | Reference | formula according to |
| | 0.1% DIFFERINE ® gel | Example 1 |
| Applied dose μg | 9.74 ± 0.65 | 9.27 ± 0.52 |
| Unabsorbed dose μg | 8.74 ± 0.76 | 6.13 ± 0.35 |
| % of the applied dose | 91% | 67% |
| Dose absorbed in | 0.11 ± 0.02 | 0.49 ± 0.11 |
| epidermis + stratum | 1.2% | 5.3% |
| corneum: SC + E μg | | |
| % of the applied dose | | |
| Dose absorbed in | 0.013 ± 0.005 | 0.05 ± 0.02 |
| dermis: D | 0.14% | 0.49% |
| % of the applied dose | | |
| (1) Dose absorbed in | 0.13 ± 0.02 | 0.54 ± 0.13 |
| the whole skin: | 1.3% | 5.8% |
| SC + E + D μg | | |
| % of the applied dose | | |
| (2) Dose absorbed in | <LQ | <LQ |
| the recipient liquid | 0% | 0% |
| % of the applied dose | | |
| (1 + 2) total amount | 0.13 ± 0.02 | 0.54 ± 0.13 |
| that penetrated μg | 1.3% | 5.8% |
| % of the applied dose | | |
| Mass balance μg | 8.86 ± 0.75 | 6.67 ± 0.39 |
| % of the applied dose | 92% | 73% |

<LQ: below the limit of quantification.

These results show that regardless of the formula tested, the adapalene is distributed mainly in the epidermis, including the stratum corneum, and to a lesser extent in the dermis.

For each formula, the amounts of adapalene recovered in the collected fluids are below the limit of quantification.

The total amount of adapalene that has penetrated (that is to say into the stratum corneum+epidermis+dermis+collected fluids) was 1.3% of the applied dose for 0.1% (w/w) DIFFERINE® gel and 5.8% of the applied dose for 0.1% adapalene cyclodextrin according to Example 1.

These results show, in a significant manner, a better release and a better penetration of the adapalene dissolved in the form of complexes with the cyclodextrins according to the invention, relative to the reference product DIFFERINE® gel, in vitro on human skin. The adapalene dissolved in the form of complexes with the cyclodextrins significantly penetrates 4 times more into the skin than the reference product DIFFERINE® gel.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A cosmetic/pharmaceutical composition comprising a)adapalene and b) at least one cyclodextrin and/or derivative thereof, formulated into a physiologically acceptable aqueous medium therefor.

2. The cosmetic/pharmaceutical composition as defined by claim 1, comprising a homogeneous solution.

3. The cosmetic/pharmaceutical composition as defined by claim 1, said adapalene a) forming an inclusion complex with said b) at least one cyclodextrin and/or derivative thereof.

4. The cosmetic/pharmaceutical composition as defined by claim 1, comprising from 1% to 60% (w/w) of cyclodextrins and/or cyclodextrin derivatives.

5. The cosmetic/pharmaceutical composition as defined by claim 1, comprising from 0.01% to 1% (w/w) of adapalene.

6. The cosmetic/pharmaceutical composition as defined by claim 1, comprising about 0.1% (w/w) of adapalene.

7. The cosmetic/pharmaceutical composition as defined by claim 1, comprising about 0.3% (w/w) of adapalene.

8. The cosmetic/pharmaceutical composition as defined by claim 1, wherein the adapalene is either present alone, or in association with another active agent, or in combination with another active agent.

9. The cosmetic/pharmaceutical composition as defined by claim 1, wherein the cyclodextrins are selected from the group consisting of α-cyclodextrins, β-cyclodextrins, γ-cyclodextrins and derivatives thereof.

10. The cosmetic/pharmaceutical composition as defined by claim 9, comprising cyclodextrin derivatives selected from the group consisting of methyl derivatives of cyclodextrins, 2-hydroxypropyl-β-cyclodextrin, 2-hydroxyethyl-β-cyclodextrin, 2-hydroxypropyl-γ-cyclodextrin and 2-hydroxyethyl-γ-cyclodextrin.

11. The cosmetic/pharmaceutical composition as defined by claim 1, further comprising a basifying agent.

12. The cosmetic/pharmaceutical composition as defined by claim 1, further comprising one or more ingredients selected from the group consisting of:
a) one or more gelling agents;
b) one or more surfactants;
c) one or more fatty phases;
d) one or more preservatives; and
e) a humectant/emollient.

13. The cosmetic/pharmaceutical composition as defined by claim 1, in aqueous form.

14. The cosmetic/pharmaceutical composition as defined by claim 13, in the form of a gel, a cream-gel, a cream, an emulsion, a lotion, a spray or a mousse.

15. The cosmetic/pharmaceutical composition as defined by claim 12, comprising:
from 0.05% to 1% of adapalene;
from 3% to 40% of one or more cyclodextrins and/or derivatives thereof;
from 0.01% to 2% of one or more gelling agents;
from 0% to 1% of one or more basifying agents; and
from 0.01% to 2% of one or more preservatives.

16. The cosmetic/pharmaceutical composition as defined by claim 15, comprising:
from 0.1% to 0.3% of adapalene;
from 5% to 15% of one or more cyclodextrins and/or derivatives thereof;
from 0.1% to 1.5% of one or more gelling agents;
from 0.02% to 0.5% of one or more basifying agents; and
from 0.01% to 1.5% of one or more preservatives.

17. The cosmetic/pharmaceutical composition as defined by claim 1, formulated as a medication.

18. A process for preparing the cosmetic/pharmaceutical composition as defined by claim 1, comprising the following steps:
i) mixing a solution of one or more cyclodextrins and/or cyclodextrin derivatives with at least one compound selected from adapalene, or one of its precursors or derivatives, to obtain a homogeneous solution;
ii) preparing the formulation base; and
iii) introducing the solution obtained in i) into the formulation base prepared in ii), and obtaining a homogeneous solution.

19. The process as defined by claim 18, wherein the step i) comprises the following steps:
1) preparing a homogeneous solution of cyclodextrins with purified water;
2) introducing adapelene or one of its precursors or derivatives into the preceding solution; and
3) stirring the mixture until a homogeneous solution is obtained.

20. The process as defined by claim 18, wherein the step iii) is terminated by the following steps:
1) adding water if necessary to compensate for any losses due to evaporation; and
2) homogenizing and packaging said solution.

21. The process as defined by claim 18, wherein one or more preservatives are introduced after a homogeneous solution has been obtained in step iii).

22. The process as defined by claim 18, wherein the step i) begins by mixing a solution of cyclodextrins and/or derivatives in a basifying agent solution.

23. A regime or regimen for treating and/or preventing dermatological conditions, for treating dermatological conditions linked to a keratinization disorder involving cell differentiation and proliferation, for treating acne vulgaris, comedonal or polymorphic acne, acne rosacea, nodulocystic acne, acne conglobata, senial acne, secondary acne, solar, drug or occupational acne, comprising administering to an individual in need of such treatment, a thus effective amount of the cosmetic/pharmaceutical composition as defined by claim 1.

24. The regime or regimen as defined by claim 23, comprising topically applying said cosmetic/pharmaceutical composition onto the affected skin area of said individual in need of such treatment.

25. A non-therapeutic cosmetic treatment regime or regimen for beautifying the skin and/or improving its surface appearance, comprising topically applying the cosmetic/pharmaceutical composition as defined by claim 1 onto the skin and/or appendages of an individual in need of such treatment.
